(19) United States
(12) Patent Application Publication
(10) Pub. No.: US 2020/0037953 A1
(43) Pub. Date: Feb. 6, 2020

(54) SYSTEM, METHOD AND APPARATUS FOR MEASURING, CLASSIFYING AND DISPLAYING ELECTRICAL CARDIAC ACTIVITY

(71) Applicant: Daniel ARAGON HAN, Zapopan (MX)

(72) Inventors: Daniel ARAGON HAN, Zapopan (MX); Yoku SASHIDA MENDEZ, Zapopan (MX); Alan FRIDMAN LANDEROS, Zapopan (MX)

(21) Appl. No.: 16/471,120

(22) PCT Filed: Sep. 27, 2017
(86) PCT No.: PCT/MX2017/000111

§ 371 Date: Jun. 19, 2019

(30) Foreign Application Priority Data
Sep. 19, 2017 (MX) MX/A/2017/012013

(51) Int. Cl. A61B 5/00; G08B 7/06; G06F 21/60; A61B 5/0428; A61B 5/0432; A61B 5/0404; A61B 5/044; A61B 5/024; G16H 40/67

(52) U.S. Cl. CPC A61B 5/681; H04L 67/42; G06F 21/602; A61B 5/04282; A61B 5/0022; A61B 5/0432; A61B 5/0404; A61B 5/044; A61B 5/684; A61B 5/7225; A61B 5/7267; A61B 5/7278; A61B 5/024; A61B 5/7475; A61B 5/7405; G16H 40/67; G08B 7/06

(57) ABSTRACT

The invention relates to a system for measuring, classifying and displaying electrical cardiac activity, characterized in that it comprises a device for acquiring, processing and conditioning electrical cardiac signals, which is formed by: a signal acquisition module comprising more than two sensors for measuring electrical cardiac activity, the sensors being designed to be put on the phalanges of the fingers of a user's hand to measure cardiac electrical activity; a signal-conditioning module; a processing and logical control module; a module for feeding back to the user; a connectivity module; and a power source module, wherein the apparatus is configured to connect to a local network that includes any technological assembly such as smartphones, tablets and computers, with the possibility of connecting to the Internet or at least to a server/client with the ability to store and process information and optimize operational algorithms of the apparatus, the apparatus being integrated in such a way that it can be carried and used in the user's hand.

### FIELD OF THE INVENTION

This invention relates to the medical industry in general, in particular to the field of medical services, such as devices for monitoring and surveillance of physiological issues related to human health. Specifically, it refers to a system, method and portable apparatus for measuring, classifying and displaying electrical cardiac activity which allows for the assessment of the heart's current condition and heart rate.

### BACKGROUND OF THE INVENTION

Millions of people die or are injured every year due to lack of timely assistance or inappropriate response. The adoption of immediate measures with proper techniques may significantly reduce deaths and injuries. First aid is not a substitute for the emergency services; however, it is a first vital step in providing a quick and efficient response to help reduce serious injuries and improve chances of survival.

The chances of surviving a sudden cardiac arrest decrease to almost zero if the patient does not receive proper medical care during the first 10 minutes of the event.

Regarding cardiac activity, there are different studies to determine and diagnose multiple cardiac issues using a variety of devices.

There are devices capable of measuring cardiac activity, like the widely known Holter monitor, which has electrodes that are connected to the chest with an adhesive and which are then connected to a recording device. A doctor may use the information captured on the Holter monitor recording device to find out if the user has a heart rate problem. However, it is bulky and uncomfortable.

Cardiac activity may also be measured by an electrocardiogram, which is a short, non-invasive test that uses electrodes attached to the chest to monitor the heart rate. However, sometimes the electrocardiogram does not detect any irregularity in the heart rate as the machine is only connected for a short period.

A device, a system and a method for measuring, classifying and displaying the electrical activity of the heart in order to immediately treat heart issues would be very valuable.

A search to determine the prior art was made, in which the following cited reference documents were found.

Patent document U.S. Pat. No. 9,113,794 B2 by Jung Ook Hong and Shelten Gee Jao Yuen dated Jun. 3, 2014 (hereinafter document D1), refers to methods and devices to activate, in an efficient manner from an energy viewpoint, heart rate monitoring based on movement and skin proximity. In addition, it provides methods to operate an LED and a photodetector aimed at obtaining an accurate reading of the adaptive heart rate. The invention presented in document D1 is aimed at efficiently activating an LED for heart rate monitoring, unlike our invention, which seeks to classify the cardiac electrical activity in healthy or diseased state, like fibrillation, and generate visual and audible feedback alerts of the emergency event.

Both our invention and the above-mentioned patent share technical characteristics, as expected in the field of electronic inventions such as: Signal acquisition means, physiological sensors, analog and digital signal conditioning means, data processing circuits, means for feeding back to the user through audio, like speakers, or visual feedback through a screen, and energy sources.

However, the physical and functional principles of the above-mentioned patent and our invention have the following differences:

1. The physical origin of the signal to be processed in document D1 is obtained through an optical measurement, an LED and a photoreceptor, with which a plethysmographic curve is estimated. On the other hand, our invention obtains its signal by the principle of oxide-reduction of a metal with its salt, with which the heart electrical activity (ECG) signal, which is not sensitive to skin color, painted nails, CO₂ intoxication or severe bleeding, is obtained.

2. In the signal conditioning part, document D1 states that its bandwidth is in the range of between 0.1 and 10 Hz, unlike our system that has a bandwidth between 0.01 and 500 Hz. This allows for a signal with a wider frequency spectrum and for the detection of anomalies in the ECG signal in the signal processing stage.

3. Document D1 in combination with intensity modulation and its movement sensors is aimed at detecting a heart attack. However, since the signal is of optical origin, other disease conditions, like fibrillation, heart arrhythmias, deviation and lengthening of PRQ wave or any PQ, ST segment, cannot be detected. This is not the case in our invention, which does allows for the knowledge of such cardiac electrical activity details and provides a classification of a diseased or healthy cardiac electrical condition.

Patent application document US20160206277 A1 by Sameer Bidichandani et al. dated Jan. 21, 2015 (hereinafter document D2) was also considered, which refers in general to techniques to determine the heart rate in a user and, specifically, heart rate measurement using multiple acoustic sensors in a portable device. The invention concerns a method for heart rate monitoring of a user, which comprises: obtaining signals from multiple acoustic sensors of a portable monitor; identifying sequential heartbeats using the obtained signals; and calculating the heart rate based on at least an interval between the sequential heart beats.

The invention presented in document D2 is aimed at, and is limited to, determining a user's heart rate through acoustic sensors, unlike our invention which seeks to classify the cardiac electric activity into healthy or diseased, like fibrillation, and generate alerts through visual and audible means of the emergency event through the electrocardiogram (ECG) signal.

Similarly, both our invention and invention in document D2 share technical characteristics, as expected in the field of electronic inventions such as signal acquisition means, sensors, analog and digital signal conditioning means, data processing circuits, visual or audio feedback means and energy sources.

However, regarding physical, chemical and functional principles, document D2 and our invention differ in the following characteristics, which require an inventive step for their implementation altogether:

1. The invention in document D2 estimates heart rate through a sound sensor, normally a microphone, placed on the wrist, unlike our invention, in which sensors such as electrodes are placed on the chest or neck to obtain the ECG. The foregoing prevents the invention in document D2 from classifying the cardiac electrical activity as diseased, like fibrillation or arrhythmia though analytical methods based on an ECG wave. It is expected that the invention in document D2 could estimate heart sounds, however, this procedure is not described in the document.

2. Regarding the signal conditioning, document D2 does not state the bandwidth, only that for a signal audible to humans it may be expected to be between 20 and 20,000 Hz. However, there is no information to assume an obvious inventive step, unlike our defined range of 0.01 to 500 Hz, since it concerns cardiac electrical activity.

Patent application US2016175683 A1 by Caroli-lames H. Carnell dated Dec. 22, 2015 (hereinafter document D3) was also found, which refers to a data analysis method for fitness and athletic training, unlike our invention, which allows for the classification of cardiac electrical activity.

Both our invention and the invention in document D3 share purely technical characteristics such as a microprocessor, an energy source and a visualization screen, in the field of electronic inventions. However, the invention in document D3 describes a technology that allows for the processing of speed, strength and pressure measurements that is totally unrelated to our invention, which processes biopotentials obtained from the electrode oxide reduction physicochemical principles and, above all, classifies the cardiac electrical activity into healthy or diseased, like fibrillation.

Although document D3 concerns a speed and inertial quantification method, neither document D1, D2 and D3 by themselves or altogether reveal the elements needed to obtain, process, monitor and classify cardiac electrical activity.

In the case of document D1, the objective of information extraction is similar since they are both aimed at obtaining physiological parameters like heart rate, but the method for obtaining the signal, processing and classification differ. Also, the characteristics proposed in D2 completely differ in the type of energy used as source of information and in the classification approach, highlighting the fact that the proposed invention is related to specific conditions in the first-aid area.

In the same way, document D3 describes a technology that is totally unrelated to our invention, since it presents a system for measuring hits in boxing training. Therefore, we can easily understand that these technological developments have totally different approaches and cannot be considered to affect inventive step since they are not similar in their approach to obtaining, treatment and classification of biopotentials like cardiac electrical activity.

As a result of the foregoing, this invention could not be inferred or deduced in an obvious or evident manner from D1, D2 or D3, neither independently by themselves nor altogether, nor being compared. Therefore, neither D1, D2 nor D3 may affect the inventive step.

### OBJECTIVE OF THE INVENTION

The main objective of this invention is to make available a novel system, method and apparatus for measuring, classifying and displaying cardiac electrical activity, which allows the classification of cardiac electrical activity into a stable or diseased state of fibrillation, in situ and without the need for the patient to be moved.

Another objective of this invention is to make available a novel system, method and apparatus for measuring, classifying and displaying cardiac electrical activity which is also portable and lightweight (500 g), below the average weight of cardiac electrical activity devices such as: heart monitors, automatic or semiautomatic defibrillators.

Another objective of this invention is to make available a novel system, method and apparatus for measuring, classifying and displaying cardiac electrical activity which, moreover, in the case of stable condition, can provide an estimate of heart rate and its bradycardia, normocardia and tachycardia indicators, which can be used when the conditions for using the heart rate monitor are not appropriate, for example, when nails are painted, under CO₂ intoxication, severe bleeding, morbid obesity, etc.

Another objective of the invention is to make available a novel system, method and apparatus for measuring, classifying and displaying cardiac electrical activity which, moreover, can provide visual and audible feedback of cardiac electrical activity, so that receipt of information and decision-making by primary health care professionals are simplified.

Another objective of this invention is to make available a novel system, method and apparatus for measuring, classifying and displaying cardiac electrical activity that also allows for the acquisition of the signal from the neck or the chest of the patient, following the physiological principle of Einthoven's triangle.

Another objective of this invention is to make available a novel system, method and apparatus for measuring, classifying and displaying cardiac electrical activity that also allows the acquisition of relevant information on the heart condition of the patient in emergency situations and situations of poor access.

Another objective of this invention is to make available a novel system, method and apparatus for measuring, classifying and displaying cardiac electrical activity also that also allows for a better accessibility of the primary health care professionals to acquire high-quality and functional technologies.

Another objective of this invention is to make available a novel system, method and apparatus for measuring, classifying and displaying cardiac electrical activity that also allow for the knowledge of the cardiac electrical activity condition in a faster manner, improving hospital practices.

All qualities and objectives that will become apparent when describing this invention are supported by illustrated embodiments.

### BRIEF DESCRIPTION OF THE INVENTION

Broadly speaking, the system and apparatus for measuring, classifying and displaying electrical cardiac activity comprises a device for acquiring, processing and conditioning electrical cardiac signals, which consists of a signal acquisition module, a signal-conditioning module, a processing and logical control module, a module for feeding back to the user, a connectivity module and a power source module; this apparatus is configured to connect to the Internet through a local network using any technological assembly such as smartphones, tablets and computers, etc., with the option of connecting to the Internet or at least to a server/client with the ability to store and process information and optimize algorithms. The system is integrated in such a way that it is portable and can be handheld.

The signal acquisition module consists of more than two sensors for measuring electrical cardiac activity, including but not limited to passive and active electrodes made of Ag/AgCl or other metals or materials, designed to be put on the phalanges of the patient, for measuring the cardiac electrical activity, which are directly connected to a signal conditioning module through metal wires. Such sensors are placed on the patient according to Einthoven's triangular pattern to record cardiac electrical activity signals, with the aim of obtaining a percentage of the lead projection, either I, II, III.

Sensors are directly connected to a high active impedance stage to preserve the signal-noise ratio and avoid information loss. The output of the high impedance stage is directly connected to the signal conditioning module through metal wires.

In addition, the signal acquisition module has a stage for the identification of the sensor type that is connected to the apparatus. The identification stage consists of an integrated circuit and a resistive circuit where the sensor's unique identification number is coded and stored. This circuit is directly connected to the processing and logical control module in order to be able to adjust the signal selector sampling frequency, which has a high-pass and low-pass filter effect depending on the commutation speed, and to implement a Finite Impulse Response (FIR) Filter specific to that type of sensor.

The signal conditioning module consists of an electronic circuit with a differential input with high impedance, an electrical insulation circuit, a pre-amplifier, a band-pass filter (high-pass filter and low-pass filter) and, lastly, an amplifier to obtain a signal limited in voltage and frequency. This module is directly connected to the processing and logical control module through metal tracks.

This processing and logical control module allows for obtaining information from the conditioned signal through the implementation of algorithms and the start and update of the feedback module. It is subdivided into four stages:
a. Conversion of the signal coming from the conditioning stage from analog into digital.
b. Processing of the registered signal.
c. Implementation of the algorithms for the classification of the cardiac electrical activity condition and heart rate estimate.
d. Start, control and update logic of the stage of feeding back to the user.

The module for feeding back to the user consists of: a) a sound device activated by the processing and logical control module logical stage; b) a visual device or screen for displaying the information on the electrical activity condition and heart rate. Initializing and updating of the logical stage of such processing and logical control module.

The portable power source module provides power to the signal acquisition module, the signal conditioning module, the processing and logical control module, and the module for feeding back to the user.

To better understand the characteristics of this invention, illustrative drawings described below are attached to this description as an integral part thereof.

### BRIEF DESCRIPTION OF FIGURES

FIG. 1 is a schematic diagram of the system involving the invention apparatus that interacts with a local network or server.

FIG. 2 is a block diagram of the subsystems integrating the invention system involving the invention apparatus that interacts with a local network or server.

FIG. 3 is a schematic diagram of the apparatus for measuring, classifying and displaying cardiac electrical activity, placed on the upper limb of a user.

FIG. 4 is an example of one of the multiple possible ways to implement or place the sensors on the cell tissue.

FIGS. 5, 5a and 5b are a conventional perspective view, a left lateral view and a right lateral view, respectively, of the apparatus for measuring, classifying and displaying cardiac electrical activity, together with its sensors and grips.

FIGS. 6, 6a and 6b are conventional perspective, front and back views, respectively, of the apparatus for measuring, classifying and displaying cardiac electrical activity.

FIG. 7a is a block diagram of the module interconnection that integrates the apparatus for measuring, classifying and displaying cardiac electrical activity, the local network and the server.

FIG. 7b is a block diagram that illustrates the main functions of each module comprising the apparatus for measuring, classifying and displaying cardiac electrical activity, the local network and the server.

FIG. 8 is a schematic diagram of the interconnection of sensors placed on the tissue or skin and the sensor identifier, the network to average acquired signals, the selector of the signal to be filtered, the analog filtering of the signal made of pre-amplification stages, band-pass filtering and amplification, as well as the processing and logical control module.

FIG. 9 is a schematic diagram that shows the effect of the multiplexer, working as a band-pass filter in the signal conditioning, as the signal sampling speed is modified in the apparatus for measuring, classifying and displaying cardiac electrical activity.

FIG. 10 is an exemplary diagram of the inverter amplification and pre-amplification settings implemented in the instrumentation of the apparatus for measuring, classifying and displaying cardiac electrical activity.

FIG. 11 is an exemplary diagram of the non-inverter amplification and pre-amplification settings implemented in the instrumentation of the apparatus for measuring, classifying and displaying cardiac electrical activity.

FIG. 12 is an exemplary diagram of one of the low-pass filtering settings implemented in the instrumentation of the apparatus for measuring, classifying and displaying cardiac electrical activity.

FIG. 13 is an exemplary diagram of one of the high-pass filtering settings implemented in the instrumentation of the apparatus for measuring, classifying and displaying cardiac electrical activity.

FIG. 14 is an exemplary block diagram of the basic logic and control structure implemented in the microcontroller of the apparatus for measuring, classifying and displaying cardiac electrical activity.

FIG. 15 is a block diagram of the main functions of the connectivity module of the apparatus for measuring, classifying and displaying cardiac electrical activity.

FIG. 16 is a flowchart of the process of acquisition of new information up to the display of information with the apparatus for measuring, classifying and displaying cardiac electrical activity.

FIG. 17 is a flowchart of the processing of the information once it is stored, where the digital filtration is adjusted to the sensor identifier of the apparatus for measuring, classifying and displaying cardiac electrical activity.

FIG. 18 is the flowchart of information analysis, which is divided into two major areas: the first one is time analysis and the second one is time-frequency analysis.

FIG. 19 is a graph of the results and indicators obtained in the processing and analysis of the stored information.

FIG. 20 is an exemplary schematic diagram of the implementation of a neural network for the classification of the cardiac electrical activity parameters obtained with the apparatus for measuring, classifying and displaying cardiac electrical activity after the time-frequency analysis.

FIG. 21 is an exemplary graph of the implementation of a support vector machine to classify the existence of fibrillation or the absence thereof in a cardiac electrical activity signal.

FIG. 22 is an exemplary graph implementation of a fuzzy system for the classification of heart rate according to the patient's age into bradycardia, normocardia or tachycardia, where the age information is obtained from the user's feedback in the local network.

FIG. 23 is the flowchart of the interaction of the local network together with the apparatus for measuring, classifying and displaying cardiac electrical activity, up to the point where the information is exported to the server.

FIG. 24 is a flowchart of the interaction of the local network with the server, as well as the information synchronization, checks, tests and training, and optimizations of the classifiers in the server.

FIG. 25 is a flowchart of the method for the updating of the classifiers in the apparatus for measuring, classifying and displaying cardiac electrical activity.

FIG. 26 is a schematic diagram of the process used by the apparatus for measuring, classifying and displaying cardiac electrical activity when only the apparatus is turned on.

FIG. 27 is a schematic diagram of the process used by the apparatus for measuring, classifying and displaying cardiac electrical activity when the apparatus is turned on, connected to a local network and the local network has access to the server.

FIG. 28 is a schematic diagram of the process used by the apparatus, local network and servers for the downloading and updating of the classification algorithms for the apparatus for measuring, classifying and displaying cardiac electrical activity when the updating instruction is given from the local network.

For a better understanding of the invention, a detailed description of some of its embodiments will be provided, shown in the illustrative, but not limiting, drawings attached to this description.

### DETAILED DESCRIPTION OF THE INVENTION

The characteristics of the system, method and apparatus for measuring, classifying and displaying cardiac electrical activity are clearly shown in the following description and in the illustrative drawings attached, the same reference signs being used to indicate the same parts. Those skilled in the art acknowledge that alternatives or possible variations of the invention may be generated within the scope of the same claims.

The embodiments of the invention described in this document are not limiting but illustrative. The invention embodiments described should not be understood in that they are unique, preferred or to be interpreted verbatim.

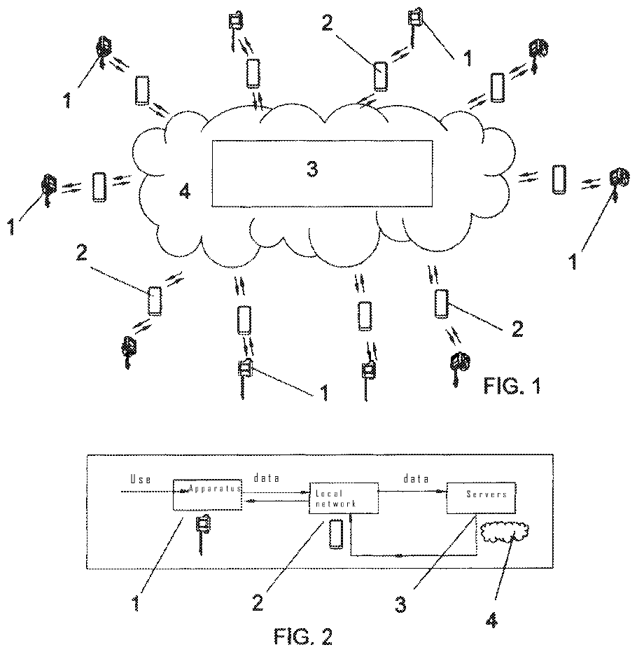

According to FIGS. 1 and 2, the system of this invention may include at least an apparatus for measuring, classifying and displaying cardiac electrical activity (1), the local networks (2) comprising any set of technology such as smartphones, tablets, computers, etc. that may be connected to the Internet or, at least, a server/client (3) in the cloud (4) with the capability of storing and processing information and optimizing algorithms.

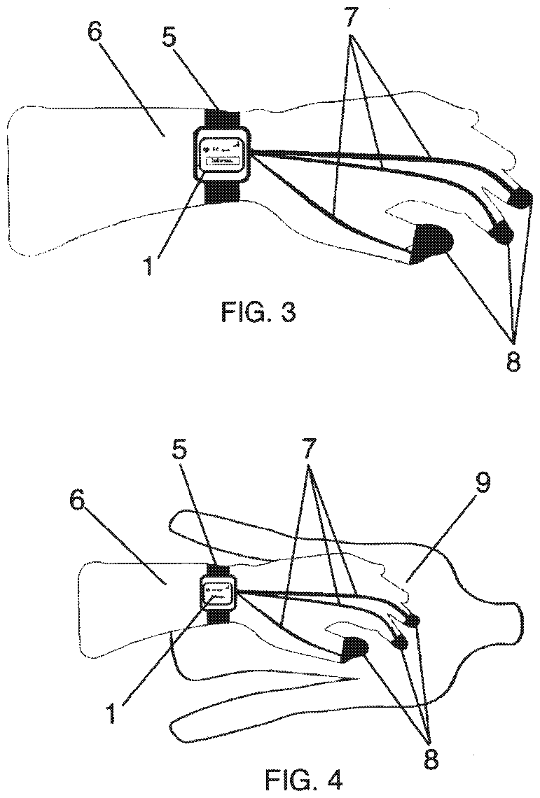

FIGS. 3 and 4 show the apparatus for measuring, classifying and displaying cardiac electrical activity (1) that comprises a band or strap (5) to attach to the user's wrist (6) and in which metal wires (7) are connected, which connect to the sensing elements (8) as passive and active electrodes made of Ag/AgCl or other metals or materials, to be put on the phalanges of the thumb, index or middle fingers of the user, for the measuring of the cardiac electrical activity, these sensing elements (8) being arranged following the pattern of Einthoven's triangle for the recording of the cardiac electrical activity signal, aimed at obtaining a percentage of the lead projection, either I, II, III, aVL, aVR, aVF.

FIG. 4 shows how the hand of the doctor holding the apparatus for measuring, classifying and displaying cardiac electrical activity (1) with the sensing elements (8) in the phalanges of the fingers is placed on the chest of a patient (9) to obtain the cardiac electrical activity signals.

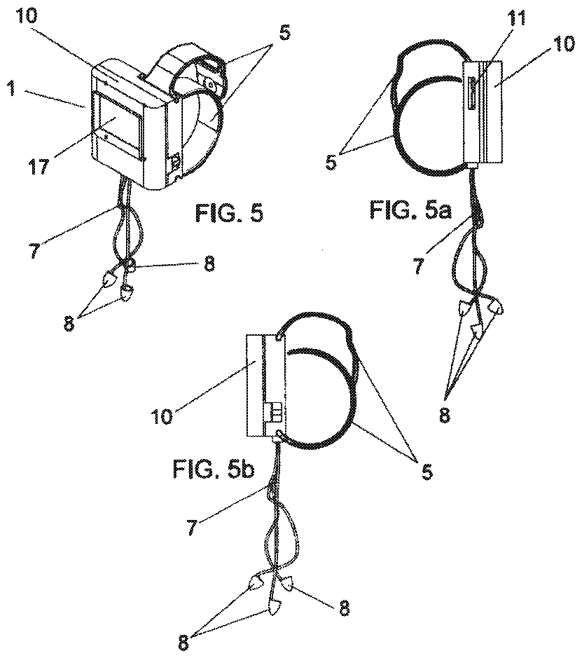

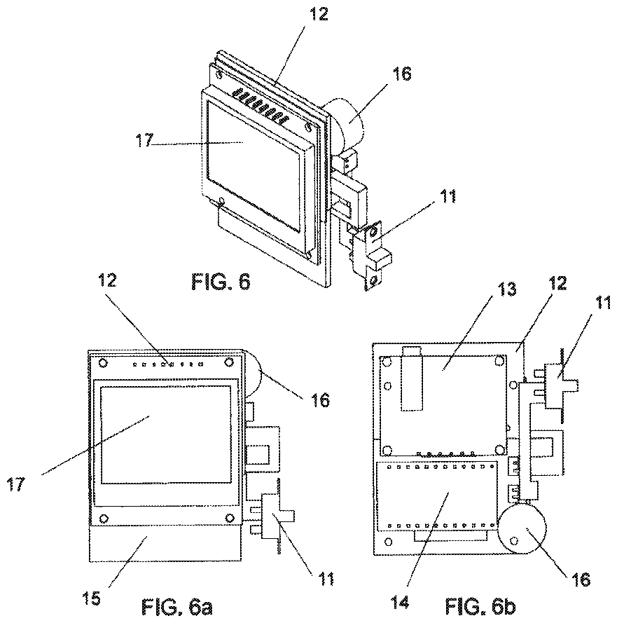

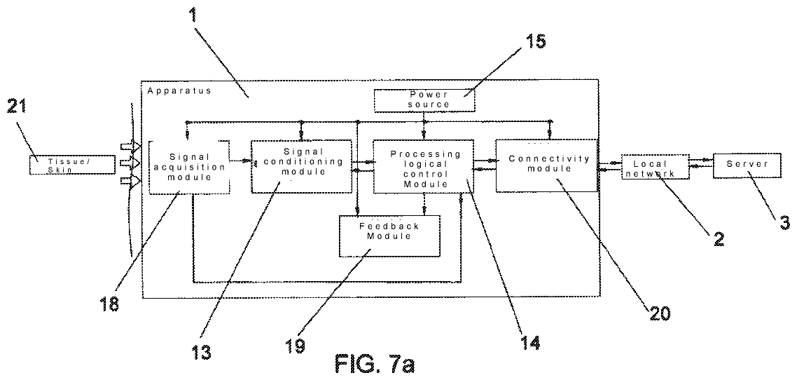

Regarding FIGS. 5 to 6b, the apparatus for measuring, classifying and displaying cardiac electrical activity (1) comprises a case (10) with the strap (5) to be attached to the user's wrist (6) and which has activation and operation buttons (11) and which internally has a support panel (12) where on one of its sides the different operation modules are arranged, indicating for these figures only those modules that are shown as the signal conditioning module (13), a processing and logical control module (14), a power source module (15), a sound source system (16) and, on the opposite side a display is placed (17).

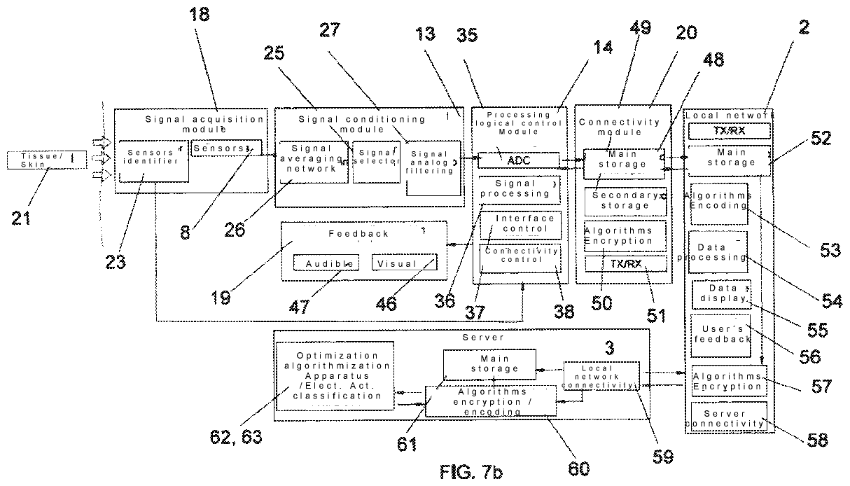

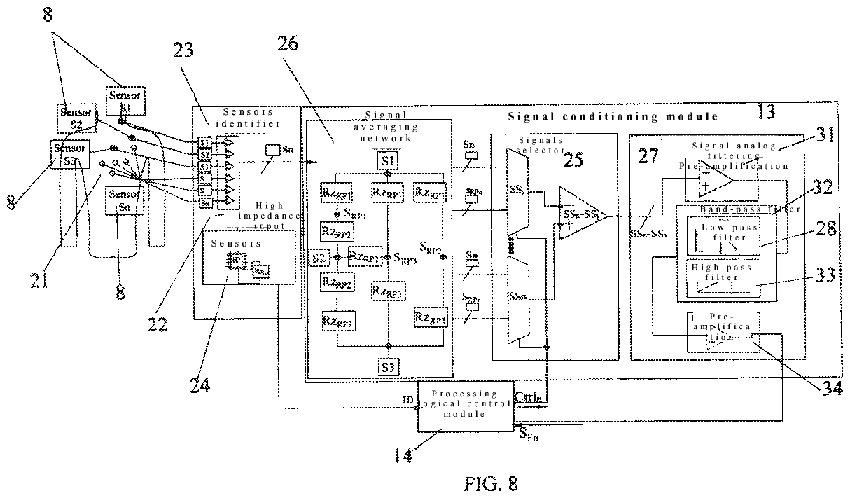

Based on FIGS. 7a, 7b and 8, the apparatus for measuring, classifying and displaying cardiac electrical activity is made of six elements that comprise a signal acquisition module (18) through which the electrical signals of the cardiac electrical activity are obtained and which has a stage for the identification of the sensor type that is connected to the apparatus; the required signals are communicated to the signal conditioning module (13) and then they go to the logical control processing module (14), a module for feeding back to the user (19) to stimulate the user's sensory channels that may include buttons, power switches, lights, displays, horns, speakers, microphones, etc.; a connectivity module (20) to communicate the apparatus to an external environment, like a local network (2) and the power source module (15) that supplies energy to the other modules.

The signal acquisition module (18) consists of two or more sensing elements (8) to measure the cardiac electrical activity, including but not limited to passive and active electrodes of Ag/AgCl or other metals or materials, to be placed on the user's phalanges. Such sensing elements (8) are placed on the tissue or skin of the patient (21) following a triangular pattern, commonly known as Einthoven's triangle, aimed at acquiring cardiac electrical activity signals. Sensing elements (8) are directly connected to a high active impedance stage (22, see FIG. 8) to preserve the signal-noise ratio and avoid information loss. The output of the high impedance stage (22) is directly connected to the signal conditioning module (13) through metal wires (7, see FIG. 3).

In addition, the signal acquisition module (18) has an identification stage of the type of sensor (23) that is connected to the apparatus. The sensor type identification stage (23) consists of an integrated circuit and a resistive circuit where the sensor unique identification number is coded and stored (24). This stage is directly connected to the processing and logical control module (14) aimed at adjusting the sampling frequency of a signal selector (25) that receives data from the signal averaging network (26) and which comprises an analog signal filtering (27), which has a low-pass filter (28) that depends on the commutation speed, and implements a finite impulse response filter specific for this type of specific sensor.

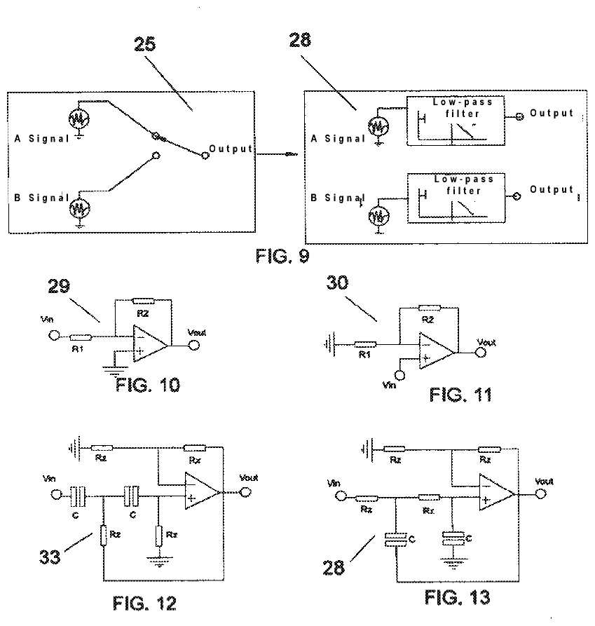

The signal conditioning module (13) consists of three stages:
a. The first stage is a signal averaging network circuit (26) to average the cardiac electrical activity obtained at the high impedance output of the signal acquisition module (18), whose main functions are to generate a mobile reference [SRP3], depending on the arrangement of the sensors on the living tissue, and to allow isolated readings of the analog combinatorics of the n-signals acquired or of the network of sensors used, like Srp1, Srp2 and Srp3 (see FIG. 8), which generate data related to the cardiac electrical activity.
b. The second stage is a high impedance signal selector circuit (25), controlled by the processing and logical control module (14). This stage is aimed at selecting the circuit input signal to the signal analog filter circuit (27) and, at the same time, applying a low-pass filter (28) that depends on the circuit commutation speed (see FIG. 9).
c. The third stage consists of a signal analog filtering circuit (27) [SSn-SSx] comprised of a pre-amplification circuit, including but not limited to a pre-amplification circuit with inverting configuration (29) or a pre-amplification circuit with non-inverting configuration (30) with a gain from 1 to 500 V/V (see FIGS. 10 and 11) and thus achieves a signal voltage adjustment [SSn-SSx]. The pre-amplification output (31) is connected to the band-pass filter input (32). This band-pass filter (32) may include but is not limited to a low-pass filter Salley-Key configuration (28) cascading with another high-pass filter Salley-Key (33) with a bandwidth from 0.005 to 500 Hz (see FIGS. 12 and 13). Lastly, the band-pass output is connected to the amplification circuit input (34). In the same way, the amplification stage may include, but is not limited to, an inverting or non-inverting configuration with a gain from 1 to 500 V/V (See FIGS. 10 and 11).

This module is directly connected to the processing and logical control module (14) through metal tracks.

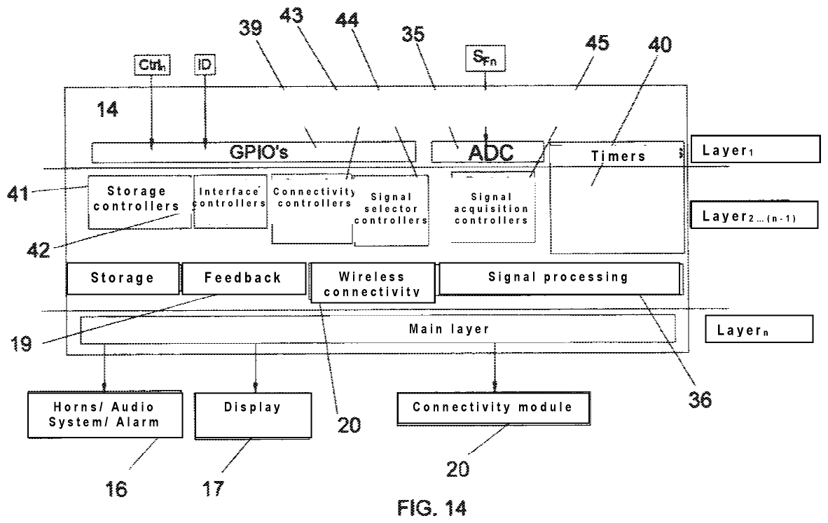

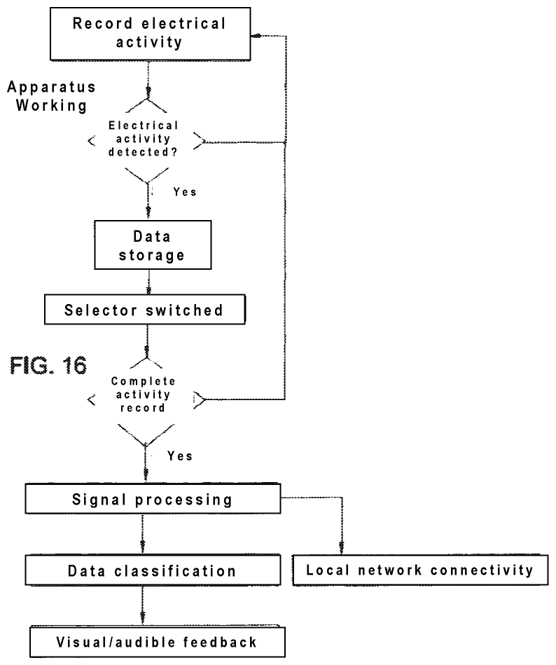

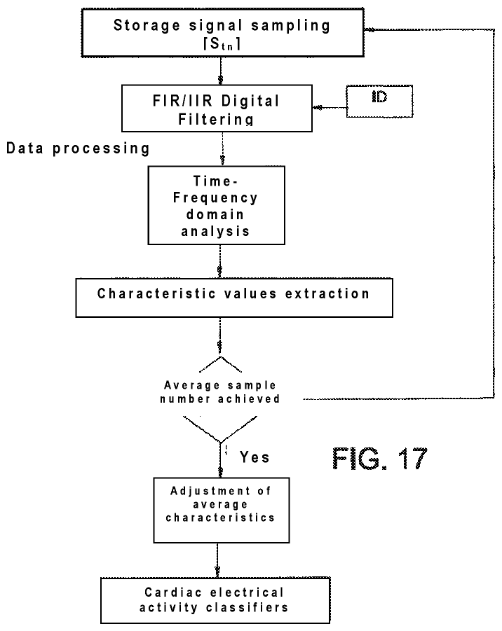

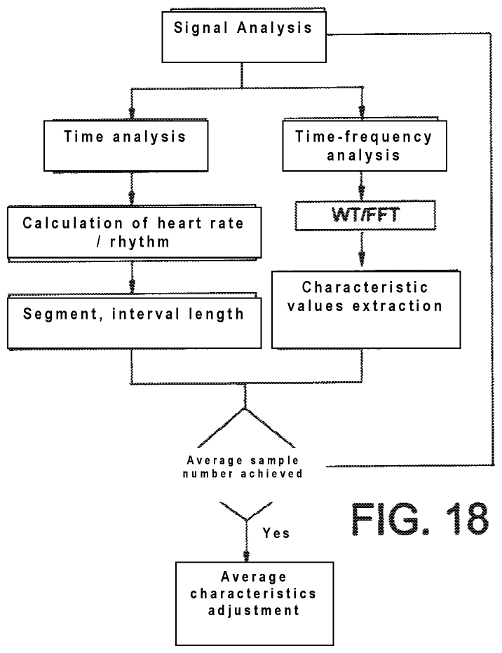

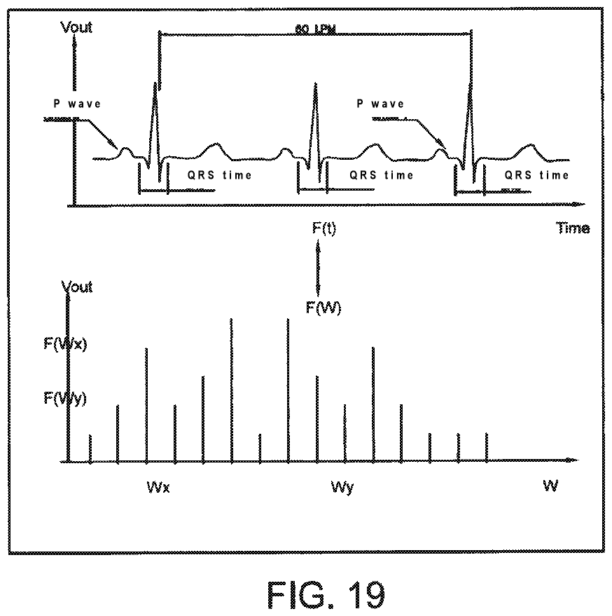

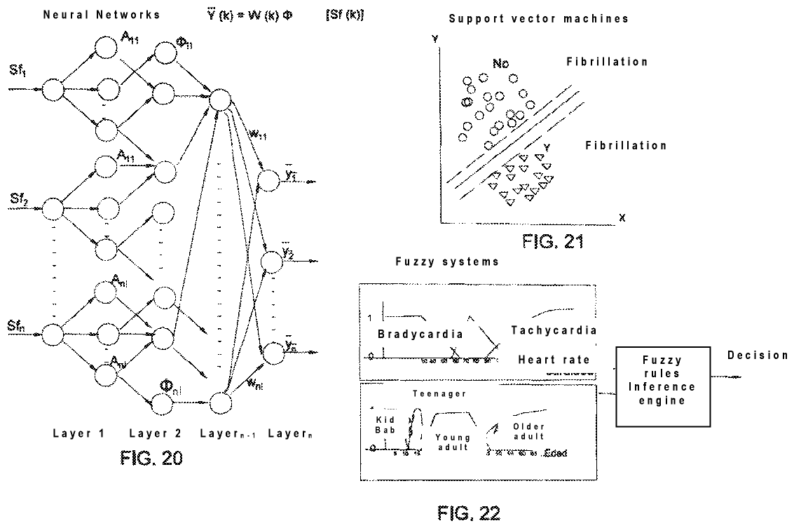

The processing and logical control module (14) is comprised of an electronic circuit that allows for obtaining a conditioned signal information through the signal conversion from analog into digital, the implementation of algorithms, including but not limited to analysis in the time-frequency and time domain, and the signal classification. Also, it starts and controls the module for feeding back to the user (19) with a display (17) and the sound supply system (16), which may be a horn for alarms, and the connectivity module (20). This module may be subdivided into four stages: (See FIGS. 7a and 14).
a. a conversion stage of analog into digital, coming from the ADC conditioning from analog to digital stage (35).
b. a recorded signal processing stage (36) including but not limited to the implementation of FIR filters associated with the sensors' ID, selector commutation speed modification, algorithm implementation for the classification of the cardiac electrical activity condition to obtain the heart rate, heart rhythm, interval or segment duration, among others. (See FIGS. 16 to 19). Some examples of the classifiers used in the apparatus can be seen in FIGS. 20 to 22, which are neural networks (FIG. 20), support vector machines (FIG. 21), fuzzy systems (FIG. 22), their combinations, like the neuro-fuzzy systems, ANFIS among others.
c. a stage of interface control (37) with initialization, control and updating logic of the stage for feeding back to the user through the user feedback module (19).
d. a stage of connectivity control (38) with initialization, control and updating logic of the connectivity module (20).

FIG. 14 is an exemplary block diagram of the logic and control basic structure implemented in the microcontroller of the apparatus for measuring, classifying and displaying cardiac electrical activity. The processing and logic control module (14) with multiple inputs/outputs for general purposes (39) and the timer block (40), as well as the different controller blocks for storage (41), interface (42), connectivity (43), signal selector (44) and signal acquisition (45) are shown.

The module for feeding back to the user (19) aims at stimulating the user's sensory channels, including but is not limited to visual (46) and audible (47) feedback, through components like: buttons, power switches, lights, LEDs, eight-segment circuits, LED, OLED and LCD displays (17, see FIGS. 5, 6 and 14), horns, speakers, microphones, among others. It is mainly connected to the processing and logical control module (14).

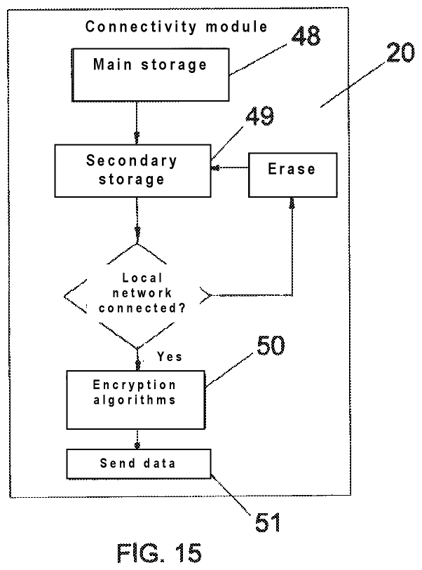

The connectivity module (20) (see FIGS. 7a, 7b and 15) is aimed at communicating the apparatus with an external environment, herein referred to as the local network. This may include, but is not limited to, sending coded or encrypted information, with a main storage (48) of information obtained thus far and a secondary storage (49) of relevant information for the device. If it is determined that such a module is connected to a local network, then encryption algorithms (50) are executed, compressing and encrypting the information to send and receive the data (51) [send (TX)/receive (RX)], if the information is not erased; in addition, it allows for the update of the apparatus classification algorithms, among others. This module is mainly controlled by the processing and logic control module (14).

The power source module (15) is portable and serves as the energy supply for the different modules, which may include, but is not limited to, batteries, rechargeable batteries, photovoltaic cells, solar cells.

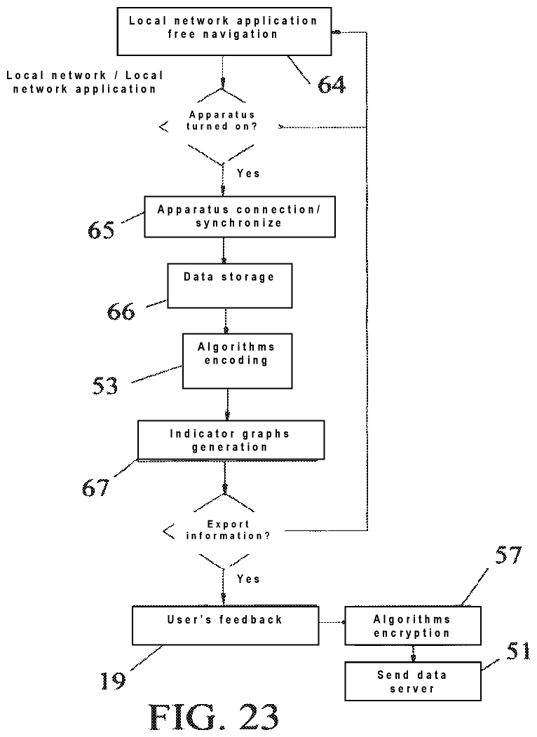

Once again, in reference to FIG. 7a, the local networks (2) to which the apparatus can connect (1, see FIGS. 5 and 6b) are aimed at exchanging information with the apparatus (1) and the server (3, see FIGS. 2, 7a and 7b), whether to send (TX)/receive (RX) and show encrypted or non-coded data or information, or to update classification algorithms. FIG. 23 shows an example of the interaction of the local network with the apparatus, where the apparatus is intended to be connected to the local network with free navigation and, if it is turned on, it is synchronized, and enables the user to extract and export information to the local network (2).

Another object of the local networks (2) is to allow for the reception of user's feedback of the information obtained with the device, such as the heart rate, any disease detected, asystole, segments increased duration, pre-infarctions or heart attacks, among others. These are used, but are not limited to be used, as supervised training mechanisms, validations, tests, optimizations, fuzzy sets, classes in the classification algorithms of the cardiac electrical activity. In the same way, when the user wishes to export the information generated up to that moment, either through printing, PDF, e-mail or another existing media, a new user's feedback is requested to validate non-sensible data for statistical purposes, such as sex, age, population, etc., which are used, without limitation, for supervised training mechanisms, validations, tests, optimizations, fuzzy sets, classes in the classification algorithms of the cardiac electrical activity. (See FIG. 23).

FIG. 7b shows that inside the local networks (2) information is transmitted or received (TX/RX), main storage is performed (52), encoding algorithms are executed (53), data processing is performed (54), data are displayed (55), user's feedback (56) is performed to validate non-sensible population data with statistical purposes, encryption algorithms are executed (57) and the server (58) connectivity with the server (3) is allowed.

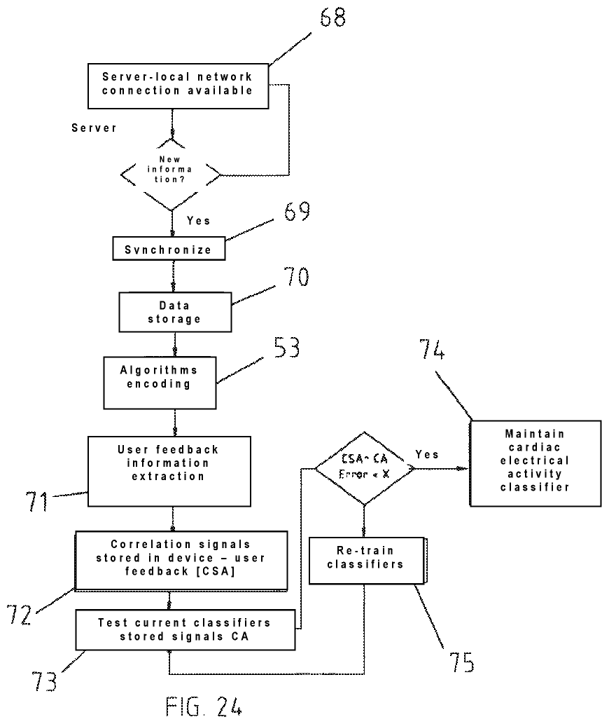

The server/client (3) on the cloud (4, see FIG. 2) offers a local network connectivity (59) with the local network (2) where the information generated by the apparatus is encrypted again with encoding and encryption algorithms (60) and is stored (61). In addition, the apparatus algorithms (62) and the cardiac electrical activity classification algorithms (63) are optimized, updated and validated. Some examples of the processes performed by the server are principal component analysis, variable correlation analysis, feedback grouping into: user's profile, population data, age, sex among others, neural networks tests (See FIG. 24). To update the device, it is first required that the device and the local network be synchronized and then the server is synchronized with the local network, where firstly the updates are download to the local network and then the apparatus is updated.

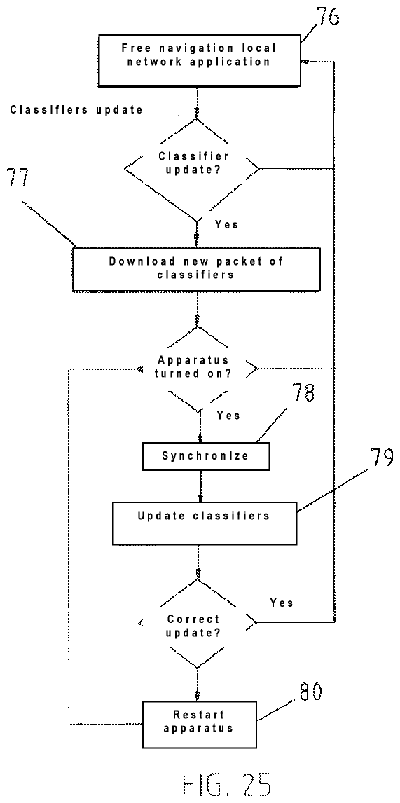

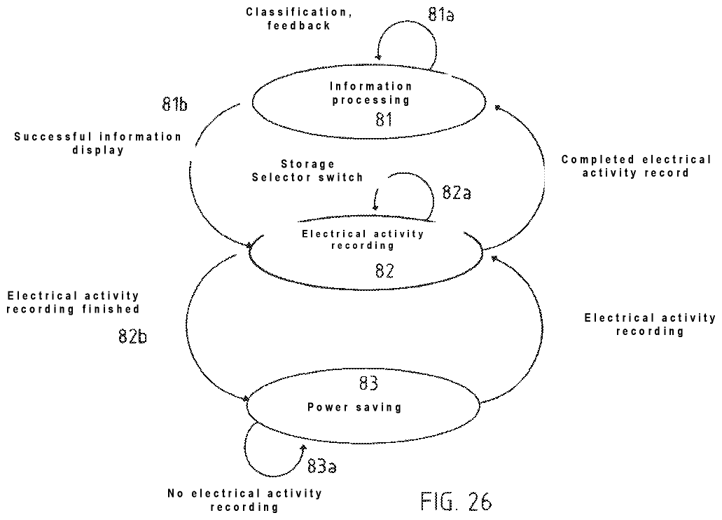

This way, with the interaction of the apparatus (1), the local network (2) and the server (3), a dynamic device capable of updating the method for measuring, classifying and displaying the cardiac electrical activity as feedback validated by the users is acquired, as shown in FIG. 25.

FIG. 23 is a flowchart of the interaction of the local network together with the apparatus for measuring, classifying and displaying cardiac electrical activity, up to the point where the information is exported to the server. It starts from the free navigation of the application in the local network (64), if the apparatus is turned on; then, it is connected and synchronized (65); after that, a data storage (66) is performed, and then encoding algorithms (53) are implemented and indicators graphs (67) are generated; if the user wishes to export information, then the user's feedback (19) is carried out, and the encryption algorithms (57) are implemented and data are sent to the server (51).

FIG. 24 is a flowchart of the interaction of the local network with the server, as well as the information synchronization, the checks, tests and training and optimizations of the classifiers in the server. In this figure, the process starts with the connection of the server to the available local network (68), if there is information, then the synchronization (69) is performed and the data are stored (70), then the encoding algorithms (53) are implemented and after that the user's feedback information extraction (71) is carried out, and then a correlation of signals stored in the device with user's feedback [CSA] (72) is executed, after that a current classifier test is carried out with stored signals [CA] (73), if [CSA]≈[CA] Error<X, then the cardiac electrical activity classifier (74) is maintained, if there is no error, a retraining of the classifier (75) is executed and the current classifier test is executed again with stored signals [CA] (73).

Regarding FIG. 25, which illustrates a flowchart of the method for updating the classifiers in the apparatus for measuring, classifying and displaying cardiac electrical activity. In this figure, a free navigation in the application of a local network (76) is observed at the beginning of the process, if there is a classifier update then a new packet of classifiers (77) is downloaded, if the apparatus is turned on, then the synchronization (78) is carried out and after that a classifier update (79) is executed and if the update is correct, the free navigation in the local network application (76) is restarted, otherwise the apparatus (80) is restarted until the update is executed.

FIG. 26 is a schematic diagram of the process used by the apparatus for measuring, classifying and displaying cardiac electrical activity only when the apparatus is turned on. This figure shows that when the apparatus is turned on and in operation it is in power saving mode (83), until it is prompted to record the activity (82), where the storage of information is performed by switching the selector (82a), when the electrical activity is no longer recorded (82b) the apparatus continues with the information processing (81) where a classification and feedback is performed (81a); if the apparatus displays the information successfully (81b) then it can resume the recording of another electrical activity (82) where the storage of information is performed by switching the selector (82a) again; when the electrical activity is no longer recorded (82b) the apparatus goes into power saving mode (83), while the apparatus is in this mode, no electrical activity reading is performed (83a).

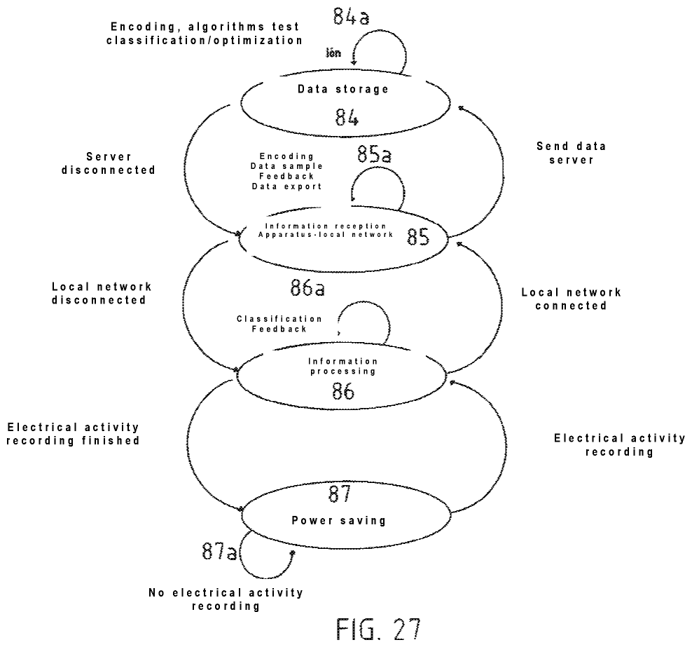

FIG. 27 is a schematic diagram of the process used by the apparatus for measuring, classifying and displaying cardiac electrical activity when the apparatus is turned on, connected to the local network and the local network has access to the server. This figure shows that the process starts when the apparatus records an electrical activity and performs information processing (86), where it is classified and feedback is performed (86a). The local network connection to the apparatus is sought for information receipt (85), where encoding is performed, data are shown, user's feedback is performed and data is exported (85a). If the delivery of data to the server is initiated in the local network, data storage (84) is generated, in this stage an encoding, classification/optimization algorithm test is performed; if the server is only connected to a local network, it receives information from the apparatus (85) carrying out an encoding, data sampling, feedback and data are exported (85a) to send the data to the server later, when it is connected; if the local network is not connected, the apparatus performs the information processing (86) and carries out classification and feedback (86a) in this case. If the local network is connected, then the local network receives information from the apparatus (85) and subsequently when the recording of the electrical activity is finished, the apparatus switches to power saving mode (87). While the apparatus is in this mode it does not perform electrical activity readings (87a), when it switches from this mode it records electrical activity again.

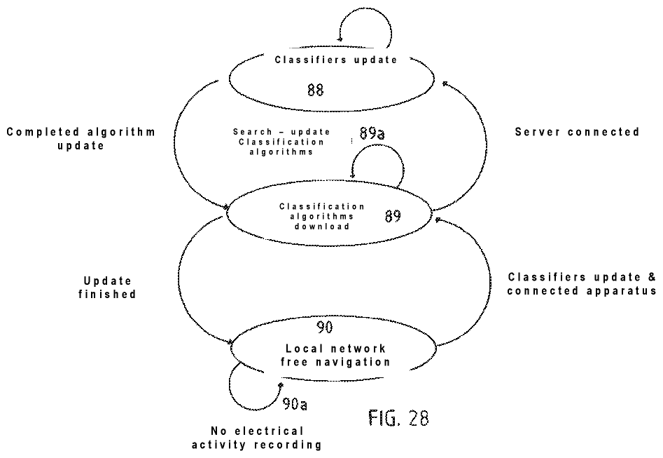

FIG. 28 illustrates a schematic diagram of the process used by the apparatus, local network and servers for downloading and updating the classification algorithms for the apparatus for measuring, classifying and displaying cardiac electrical activity when the updating instruction is given from the local network. In this figure, the process starts with the classifiers' update stage (88), when the algorithms update is completed, then the classification algorithms are downloaded (89), the apparatus performs a search and update of the classification algorithms (89a). When the apparatus finishes its update, it is free to navigate in the local network (90) without recording electrical activity (90a) and then the process starts again.

Taking into consideration FIGS. 1 to 28 and especially 16 to 28, the method used for the extraction, handling and classification of the cardiac electrical activity information comprises the following:

Classification Method:

1. The user, with the apparatus (1) on their hand, as illustrated in FIGS. 3 and 4, places the sensors on the living tissue of the chest or neck:

2. The user turns on the apparatus (1)

3. The user starts recording the cardiac electrical activity signal.
a. When the signal recording does not start, the apparatus (1) switches to power saving mode.

4. The processing and logical control module (14) identifies the ID of the sensing elements (8);

5. The processing and logical control module (14) sets the multiplexing speed of the signal selector (25);

6. The processing and logical control module (14) adjusts the parameters of the digital filters that depend on the ID of the sensing elements (8);

7. The processing and logical control module (14) adjusts the number of times that it must obtain the signal to process it depending on the ID;

8. The cardiac electrical activity recording starts;

a. Depending on the ID of the sensing element (8) and the signal selector configuration (25), the reading of a network voltage is obtained to average the signal, which goes to the signal conditioning module (13), where it is pre-amplified, filtered and amplified, to then be captured in the ADC (35) of the processing and logical control module (14).

b. The data captured by the ADC is stored (35).

c. According to the ID of the sensing elements (8), the storage of an n-number of data shall be generated. Once the storage of this signal is obtained, the signal selector (25) is switched to a different signal averaging network input (26).

9. Records another n-number of signals and switches again;

a. According to the ID of the sensing elements (8), the signal selector (25) switches for n-inputs of the signal averaging network (26);

b. Once all the n-inputs were switched, the signal record starts again until it gets to the n-number of times of signals to average according to the ID of the sensing element (8);

10. It reaches the average number of signals to be recorded;

11. The information processing starts;

a. Depending on the ID of the sensing elements (8), the processing and logical control module (14) applies digital filters (FIR/IRR) to each of the recorded signals;

b. Time and time-frequency analysis are applied to the signal to extract information characteristics of the previously stored signals;

c. Each of the indicators obtained is averaged.

12. Indicator classifiers are implemented, which also depend on the ID of the sensing elements (8) and the algorithms preset from the local network (2) and the server (3) in the apparatus (1);
a. They are either neural networks, fuzzy systems and vector support machines or their respective combinations

13. Visual (46) and audible (47) feedback is generated through the feedback module (19);
a. Results are shown in the display screen (17) as indicators: heart rate;
b. In addition, the device alarm can be activated if any classifier result needs to be reviewed in detail.

14. Once the cardiac electrical activity signal acquisition is finished and it is connected to the local network (2);
a. Data are encrypted;
b. An apparatus (1)-local network (2) data sending and receipt is generated

15. The local network (2)
a. Synchronizes the information of the apparatus (1)-local network (2);
b. Makes a copy of the current information (Primary Data);
c. Decodes the information;
d. Processes the information to present it as graphs, indicators or other display forms.

16. If the user wishes to export the information for any reason, including but not limited to: printing, sending it to a health professional;
a. The user should validate the acquired information, which is considered as users feedback, aimed at obtaining reliable data including but not limited to:
   i. Population data: sex, age, nationality, origin;
   ii. Possible alterations in the cardiac electrical activity observable by the health professional: Asystole, fibrillation, atrial, ventricular, etc.
   iii. Users profile: Physician, nurse, paramedic, years of experience;
b. The local network (2) compresses, encrypts and stores the users feedback plus the primary data to obtain an information package;
c. The local network (2) exports the signal to any possible media;

17. If it is connected to the server, then
a. It synchronizes the local network (2) with the server (3)
b. It transfers the data packet;
c. It makes a copy of the data packet;
d. It decrypts the data packet;
   i. It uses the primary data to:
      1. Validate the classification algorithms, either the neural networks, fuzzy systems, support vector machines
      2. Test the classification algorithms
      3. Generate, retrain or optimize the classification algorithms of the cardiac electrical activity signal, comparing them to the reference mean error
   ii. Uses the users feedback to:
      1. Correlate the primary data with the users feedback (CSA) to create clusters of words and observations and use them
      2. Validate, test and retrain the classification algorithms

18. For the apparatus algorithm updating (1):
a. Updating is requested from the local network (2);
b. Connection of the apparatus (1) and the server (3) is verified;
c. The updates are downloaded from the server (3) to the local network (2);
d. The algorithms are downloaded from the local network (2) to the apparatus (1);
e. The algorithms are stored in the memory of the apparatus (1); whether in the primary or the secondary memory;
f. And the memory algorithms are updated;
g. The apparatus is restarted (1);
h. It is verified if the update was correct.

Application example of the apparatus for measuring, classifying and displaying the electrical cardiac activity pursuant to this invention.

### EMBODIMENTS

An application example of the apparatus arises when a pre-hospital, emergency or primary health care professional faces an emergency situation where they need to know the patient's heart condition. The user simply turns on the apparatus (1), puts the sensing elements (8) on the neck or on the chest of the patient (see FIG. 4) and records the cardiac electrical activity. The user must wait some minutes while the signal is recorded and processed according to the identifier of the sensing elements (8), since it adjusts the above-mentioned parameters like the selector commutation speed, the application of a certain FIR, a minimum number of records taken to average the signal, among others, and the type of classifier used. Two possible combinations could be displayed: the first is a healthy heart condition, where the patient's heart rate and an indicator of bradycardia, normocardia or tachycardia may be shown. The second is a diseased condition, where an alarm and the indicator of the diseased condition of the cardiac electrical activity such as asystole and atrial or ventricular fibrillation are shown. Thus, the health professional, based on their experience and the information provided by the apparatus (1), shall follow the proper protocols for the situation. As shown in FIG. 26.

A second example may be that the health professional synchronizes the apparatus (1) with the local network (2) such as their cellphone and sees the graphs and indicators extracted from the analysis of the record. Finally, the health professional will have the option, depending on the time, the severity of the situation and the availability of equipment, to export the information for their personal use. In this way, they will contribute to the generation of validated information. As shown in FIG. 27.

For cases where the apparatus (1) was turned on but there is no instruction to record cardiac electrical activity, the apparatus (1) will remain in this power saving mode until it turns off or an instruction to record the cardiac electrical activity is generated.

Sometimes, not in critical moments and under the responsibility of the user, the updating of the apparatus (1) classification algorithms can be managed, as shown in FIG. 28.

The main improvements of the system include the following:

1. It is a portable system, it can be carried in the hand, kept in a backpack or an ambulance, the weight is below the average weight of the cardiac electrical activity measurement devices like: cardiac monitors, automatic or semi-automatic defibrillators, of approximately 3.3 kg compared to the 500 g of this system.

2. It extracts basic parameters of the electrical activity condition, including but not limited to: Heart rate, heart rhythm, interval duration and multiple segments like the QRS complex, ST segment, T wave.

3. It classifies the cardiac electrical activity into stable and diseased conditions, like fibrillation, in situ not needing to transfer the patient.

4. In the case of a stable condition, it provides an estimate of the heart rate and its indication of bradycardia, normocardia or tachycardia.

5. It provides visual and audible feedback of the cardiac electrical activity so that the receipt of information and the decision-making by the first-contact health professionals are simplified.

6. The placement and instrumentation of the modules allows the acquisition of the signal from the patient's neck or chest, following the physiological principle of Einthoven's triangle.

7. The system operation method allows for updating the classification algorithms of the apparatus.

8. The system operation method allows for updating of extraction algorithm characteristics.

9. The system allows the tracking and identification of the apparatus.

10. The system allows for the identification of the types of sensors used for the recording of the cardiac electrical activity and thus the processing and conditioning of the recorded signals, the multiplexor sampling frequency and the bandwidths of the FIR can be adjusted.

11. It allows the export of the information to a local network (2) like cellphones, tablets, computers, laptops for the display and handling of the information by the user.

12. The specialist's feedback together with their specialist profile allows the extraction of information for the training, testing and validation of the classifiers, either neural networks, support vector machines, fuzzy systems or any other possible method in the area of artificial intelligence or deep learning machines.

13. It allows for the creation of medical condition models related to the cardiac electrical activity from the analysis of the information extracted from the records and the database.

The invention has been sufficiently described so that a person with average knowledge in the field may be able to replicate it and obtain the results mentioned herein. However, any expert in the field of this invention may be able to make modifications not described herein; however, if in the application of these modifications in a specific structure or in its manufacturing process, the subject matter claimed in the following claims is required, such structures shall be comprised within the scope of this invention.

After a thorough description of the invention, it is considered as novel and, therefore, what is expressed and contained in the following claim clauses is claimed as property:

1. A system for measuring, classifying and displaying electrical cardiac activity characterized in that it comprises an apparatus for acquiring, processing and conditioning electrical cardiac signals, which consists of a signal acquisition module, a signal-conditioning module, a processing and logical control module, a module for feeding back to the user, a connectivity module and a power source module; this apparatus is configured to connect to a local network including any technological assembly such as smartphones, tablets and computers, with the possibility of connecting to the Internet or at least to a server/client with the ability to store and process information and optimize operational algorithms; this apparatus is integrated in such a way that it is portable and handheld.

2. The system for measuring, classifying and displaying electrical cardiac activity, according to claim 1, characterized in that such a signal acquisition module comprises more than two sensors for the measurement of the cardiac electrical activity, designed to be placed on the phalanges of the user's fingers, for measuring the cardiac electrical activity of the patient, which are directly connected to a stage of high active impedance that preserves the signal-noise ratio and avoids information loss; where the high impedance stage output is directly connected to the signal conditioning module through metal wires.

3. A system for measuring, classifying and displaying electrical cardiac activity according to claim 2, characterized in that such sensors are placed on the tissue or skin of the patient following the pattern of Einthoven's triangle for the recording of the cardiac electrical activity signal, in order to obtain a percentage of the lead projection, either I, II, III, aVL, aVR, aVF.

4. A system for measuring, classifying and displaying electrical cardiac activity according to claims 1 to 3, characterized in that such signal acquisition module has a sensor type identification stage that is connected to the apparatus made of an integrated circuit and a resistive circuit where the sensor unique identification number is coded and stored; such stage is directly connected to the processing and logical control module aiming at adjusting the sampling frequency of a signal selector, which has a low-pass filter and high-pass filter that depends on the commutation speed, and implements a finite impulse response filter (FIR) specific to this type of sensor.

5. A system for measuring, classifying and displaying electrical cardiac activity according to claim 1, characterized in that such signal conditioning module is made of an electronic circuit of the signal averaging network to average the cardiac electrical activity signal with a high impedance differential input, the analog filtering of the signal made of stages of pre-amplification, a high impedance signal selector circuit that applies a low-pass filter, a signal analog filtering circuit defined by a pre-amplification circuit with band-pass filters (high-pass filter and low-pass filter) and an amplifier circuit to obtain a signal limited in voltage and frequency; such module is directly connected to the processing and logical control module through metal tracks.

6. A system for measuring, classifying and displaying electrical cardiac activity according to claim 1, characterized in that such processing and logical control module consists of an electronic circuit that obtains information from the signal conditioned from analog into digital, through the implementation of algorithms, analysis in the time-frequency and time domain and signal classification, and it starts and updates the module for feeding back to the user, which is subdivided into four stages:
a. Conversion of the signal coming from the ADC conditioning stage from analog into digital;
b. Processing of the recorded signal through FIR filters, modifying the selector commutation speed, implementation of the algorithms for the classification of the cardiac electrical activity condition and estimation of the heart rate, heart rhythm, duration of intervals and segments, among others;
c. Interface control with initialization, control and updating logic of the stage for feeding back to the user through the module for feeding back to the user;
d. Connectivity control with initialization, control and updating logic of the connectivity module.

7. The system for measuring, classifying and displaying electrical cardiac activity, according to claim 1, characterized in that the module for feeding back to the user is made of: a) a sound device activated by the logical stage of such processing and logical control module; b) a visual device or screen for displaying the information on the electrical activity condition and heart rate.

8. The system for measuring, classifying and displaying electrical cardiac activity, according to claim 1, characterized in that this connectivity module is set to be connected to an external environment like a local network, to send encoded or encrypted information, with a main storage of information obtained up to that moment and a secondary storage of information relevant to the apparatus; to execute information encryption algorithms to send the data (TX)/(RX), algorithms updating; controlled by the processing and logical control module.

9. The system for measuring, classifying and displaying electrical cardiac activity, according to claim 1, characterized in that such portable power source module provides power to the signal acquisition module, the signal conditioning module, the processing and logical control module, the connectivity module and the module for feeding back to the user.

10. The system for measuring, classifying and displaying electrical cardiac activity, according to claim 1, characterized in that such apparatus and local network are set to transmit and receive information; such a local network is set to store information, execute encoding algorithms, process data, display data, feedback the user, execute algorithms and connect to a server.

11. The system for measuring, classifying and displaying electrical cardiac activity, according to claim 1, characterized in that such a server is set to encrypt data encoding and encryption algorithms, store information, optimize, test, update and validate algorithms of the apparatus and of cardiac electrical activity classification, and to execute processes like variable correlation, grouping of the feedback to the user and apparatus update, among others.

12. The system for measuring, classifying and displaying electrical cardiac activity, characterized in that it has a case with the strap to be attached to the user's wrist and has activation and operation buttons and internally has a support panel where a signal acquisition module is placed through which electrical signals of the cardiac electrical activity are obtained through sensing elements placed on the phalanges of the thumb, index and middle fingers of the user and connected with metal wires to such signal acquisition module and which presents a stage of identification of the type of sensor that is connected to the apparatus; such acquired signals are communicated to a signal conditioning module to go then to a processing and logical control module, a module for feeding back to the user to stimulate the user's sensory channels which may include buttons, switches, lights, displays, horns, speakers, microphones, among others; a connectivity module to communicate the apparatus with an external environment such as a local network and a power source module that supplies energy to the other modules.

13. A method for extraction, handling and classification of the information on the cardiac electrical activity through an apparatus with an signal acquisition module through sensors placed on the user's phalanges that integrates a signal conditioning module, a processing and logical control module, a module for feeding back to the user, a connectivity module and a power source module that comprises the following stages:
1. place the sensors of the apparatus attached to the user's wrist on the chest or neck of the living being tissue;
2. turn on the apparatus;
3. start the recording of cardiac signal activity, through a signal acquisition module, otherwise the apparatus switches to power saving mode;
4. identify the ID of the sensors through a processing and logical control module of the apparatus;
5. set the multiplexing speed of the signal selector through such processing and logical control module;
6. adjust the parameters of the digital filters that depend on the ID of the sensor elements through the processing and logical control module;
7. adjust the number of times that it must get the signal to process it depending on the ID, through the processing and logical control module;
8. start the cardiac electrical activity record;
a. obtain the reading of a network voltage to average the signal (depending on the ID of the sensor element and the signal selector configuration), which goes to the signal conditioning module, where it is pre-amplified, filtered and amplified, to be captured in an ADC of the processing and logical control module;
b. store the data captured by an ADC;
c. generate a storage of n-number of data according with the ID of the sensing elements, switch the signal selector to a different signal averaging network input until getting the storage of this signal;
9. record another n-number of signals and commute again;
a. switch for n-inputs of the signal averaging network according to the ID of the sensor elements;
b. restart the signal recording until getting to the n-number of times of signals to average according to the ID of the sensor elements;
10. reach the average number of signals to be recorded;
11. start the information processing;
a. apply digital filters (FIR/IRR) to each of the recorded signals, depending on the ID of the sensor elements, through the processing and logical control module;
b. apply time and time-frequency analysis to the signal to extract information characteristics of the signals previously stored;
c. adjust by average each of the indicators obtained;
12. implement indicator classifiers which also depend on the ID of the sensing elements and the algorithms preset from the local network and a server in the apparatus;
a. either neural networks, fuzzy systems and vector support machines or their respective combinations;
13. generate visual and audible feedback through the feedback module;
a. show results in a display as indicators; heart rate;
b. activate the device alarm if any classifier result needs to be reviewed in detail;
14. encrypt data and generate data delivery and receipt from the apparatus to the local network, once the cardiac electrical activity signal recording is finished, the apparatus being connected to the local network.

14. The method used for the extraction, handling and classification of the cardiac electrical activity, according to claim 13, characterized in that for the apparatus being in communication with the local network, the following stages are executed:
15. execute in the local network
a. Synchronization of the apparatus information;
b. make a copy of the current information (Primary Data);
c. decode the information;
d. process the information to present it as graphs, indicators or other display forms;
16. export the information;
a. validate the acquired information (user's feedback) to obtain reliable data:
i. population data: sex, age, nationality, origin;
ii. possible alterations in the cardiac electrical activity observable by the health professional: Asystole, origin, auricular, ventricular;
iii. user's profile: Physician, nurse, paramedic, years of experience;
b. compress, encrypt and store the user's feedback plus the primary data to obtain an information packet;
c. export the signal to any possible media.

15. The method used for the extraction, handling and classification of the cardiac electrical activity, according to claim 14, characterized in that for the local network being connected to a server, then it is possible to:
17. synchronize the local network with the server
b. transfer the data packet;
c. make a copy of the data packet;
d. decrypt the data packet;
i. use the primary data to:
1. validate the classification algorithms, either the neural networks, fuzzy systems, support vector machines;
2. test the classification algorithms;
3. generate, retrain or optimize the classification algorithms of the cardiac electrical activity signal, comparing them to the reference mean error
ii. use the user's feedback to:
1. correlate the primary data with the user's feedback (CSA) to create clusters of words and observations and use them
2. validate, test and retrain the classification algorithms.

16. The method used for the extraction, handling and classification of the cardiac electrical activity, according to claims 13 to 15, characterized in that the apparatus algorithms update is comprised by the following stages:
18 requesting update from the local network;
a. verifying the connection of the apparatus and the server;
c. downloading updates from the server to the local network;
d. downloading the algorithms from the local network to the apparatus;
e. storing the algorithms in the memory of the apparatus, whether in the primary or the secondary memory;
f. updating the memory algorithms;
g. restarting the apparatus;
h. verifying that the update was correct.
